(19) United States
(12) Patent Application Publication
(10) Pub. No.: US 2003/0064948 A1
(43) Pub. Date: Apr. 3, 2003

(54) Invasomes for therapy of disorders, their preparation and use

(76) Inventors: Alfred Fahr, Colbe (DE); Rolf Muller, Marburg (DE)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/071,509

(22) Filed: Feb. 8, 2002

(30) Foreign Application Priority Data
Feb. 8, 2001 (DE) 10105659.1

(51) Int. Cl. A61K 48/00; A61K 9/127

(52) U.S. Cl. 514/44; 424/450

(57) ABSTRACT

The invention relates to invasomes comprising a lipid mixture comprising one or more lipids, preferably neutral lipids, one or more lysophosphatides and at least one pharmacological agent, preferably an immunomodulator, to the preparation thereof and to the use thereof for the therapy of disorders, preferably of disorders which can be treated by modulation of the immune system.

### FIELD OF THE INVENTION

The invention relates to invasomes comprising a lipid mixture comprising one or more lipids, preferably neutral lipids, one or more lysophosphatides and at least one pharmacological agent, preferably an immunomodulator, to the preparation thereof and to the use thereof for the therapy of disorders, preferably of disorders which can be treated by modulation of the immune system.

### PRIOR ART

Topical application of hydrophilic or hydrophobic pharmacologically active substances with the aim of transporting the particular agent into deeper layers of skin or even through the skin is a difficult undertaking because the topmost layer of the skin (stratum corneum) is impermeable to most substances. However, it would be desirable for many agents to be administered directly through the skin. For example, transdermal administration of insulin is of interest for diabetics because this would dispense with the daily injections of insulin. Whereas such efficient transdermal transport is desirable for all substances intended to display a systemic action, it is unsuitable for substances whose action is to be confined to the deeper layers of the skin. This applies for example to agents which show severe side effects and/or toxicity on systemic administration but nevertheless are very suitable for the therapy of certain skin disorders.

A large number of attempts to overcome the barrier of the skin has been described in the literature. Particular attention has been directed at liposomes in this connection. Mezei and Gulasekharam (Life Sci. 26: 1473-1777, 1980) were the first to report that liposomes loaded with triamcinolone acetonide made a 3- to 5-fold accumulation of the agent possible in the epidermis and dermis of the skin. Vanlerberghe et al. (Coll Nat. CRNS: 938-942, 1978) showed that the use of vesicles of nonionic surface-active substances (niosomes) increased the penetration of sodium pyrrolidonecarboxylate through the stratum corneum. WO 92/03122-A1 disclosed the use of “transferosomes” for systemic administration of insulin. The transferosomes consist of a lipid portion and of an edge-active substance. The transferosomes are also referred to as “flexible” liposomes which, in contrast to liposomes with more rigid structures, are able to facilitate the route of substances through the stratum corneum. However, these attempts were primarily aimed at transporting the particular agents as efficiently as possible through the skin into the body.

It is also desirable in the case of skin disorders involving cells in deep-lying layers of the skin, such as, for example, cells in the stratum germinativum, to transport agents efficiently through the barrier of the stratum corneum. Skin disorders involving the immune system of the skin are an important example thereof. The immune system of the skin is formed on the one hand by the cells already present in the skin, such as, for example, dendritic cells, but also by the immune cells infiltrating into the skin in each case of illness, infection and/or inflammatory processes, such as, for example, T cells, Langerhans cells and macrophages. In these skin disorders there is found to be, for example, an increase by comparison with healthy skin in the infiltration or activation of said immune cells without an injury or infection of the skin in fact being present. It is suspected that many of these disorders, such as, for example, alopecia areata, psoriasis or neurodermatitis, are also caused by autoimmune responses.

Alopecia areata is a relatively common disorder for which there is a lifetime probability of an outbreak of 1.7% in the population (Safavi et al. Mayo Clin. Proc. 70: 628-633, 1995). Alopecia areata is associated with a reversible hair loss which is confined to limited areas, but may sometimes also lead to complete hair loss (alopecia totalis). Histopathological characteristics of alopecia areata comprise perifollicular lymphocytic infiltration affecting the anagen hair follicles with subsequent miniaturization of these follicles (Golnik and Orphanos, in Orphanos and Happle (editors) Hair and hair diseases. Berlin, Springer Verlag, pages 529-569, 1990). These infiltrates consist primarily of T4 lymphocytes, macrophages and Langerhans cells (Tobin et al. J. Invest. Dermatol. 109: 329-333, 1997). The hair loss is associated with peri- and intrafollicular inflammatory infiltration of the anagen hair follicles, the infiltrate consisting primarily of CD4⁺ and CD8⁺ cells. The function of each of these cells in the pathogenesis of alopecia areata is not yet understood (Mc Elwee et al. Br.J. Dermatol. 140: 432-437, 1999). The observed peribulbar and intrabulbar accumulation of T lymphocytes (Perret et al., Acta Derm. Venereol. 64: 26-30, 1984) and dysregulation of expression of the intercellular adhesion molecule 1 (ICAM-1) and the HLA-DR molecule on follicular keratinocytes and dermal papillae (Hamm et al., Arch. Dermatol. Res. 280: 179-181, 1988; Nickoloff & Griffiths, J. Invest. Dermatol. 96: 91-92, 1991) indicate, however, that the immune system of the skin is involved in the disorder (Baadsgaard J. Invest. Dermatol. 96: 89-90, 1991).

Suitable for the treatment of these skin disorders are therefore immunomodulating substances which, for example, suppress the immune response. Such substances, which have to date been used in particular for systemic treatment of these skin disorders, are, for example, cyclosporin A or FK 506. The disadvantage of systemic administration of immunomodulatory substances is, for example in the case of cyclosporin A, the nephrotoxicity, a side effect which can be tolerated when cyclosporin is used to prevent transplant rejection reactions but not in the treatment of skin disorders which are not directly life-threatening.

The attempts described in the prior art to avoid systemic administration of immunosuppressants by topical application have not been successful to date. The methods used however led to inadequate transport through the stratum corneum or led to an unwanted increase in the serum concentration of the agent, i.e. systemic effect. For example, Griffiths et al. (Lancet I: 806, 1987) showed that a solution prepared from 2% cyclosporin A in unguentum Merck led to no effect on the treatment of psoriasis. Black et al. (J. Invest. Dermatol. 5: 644-648, 1990) used besides cyclosporin administered systemically an oily vehicle loaded with cyclosporin in order to reduce the T-cell-mediated immune response in the skin. They observed a reduced T-cell-mediated immune response but also observed an increased systemic amount of cyclosporin after termination of the systemic administration and exclusively topical administration. In the same way, Gilhar et al. (Acta Derm. Venereol., 69: 252-253, 1989) found no effect with a 10% cyclosporin A solution in an oily preparation on alopecia areata, but also found no increase in the cyclosporin level in the blood of the patients. Niemiec et al. (Pharm. Res. 12: 1184-1188, 1995) used three different nonionic liposomes and investigated the transport of cyclosporin A in a hamster ear model. Deposition of cyclosporin A was observed only on use of liposomes consisting of glyceryl dilaurate/cholesterol/polyoxyethylene-10-stearyl ether in the mixing ratio 57:15:28% by weight. A phospholipid-based liposome preparation led to only minimal uptake of cyclosporin A into the skin.

Guo et al. (Int. J. Pharm. 194: 201-207, 2000) used the transferosomes described by Cevc et al. (Adv. Drug. Del. Rev. 18: 349-378, 1996 and in WO 92/03122) in order to transport cyclosporin through the skin. However, a serum concentration of 63 ng/ml cyclosporin was observed only 2 hours after administration of the transferosomes in this study. The vesicles used by Guo et al. were prepared from high-purity lecithin and the surfactant sodium cholate. The authors draw the conclusion from the results that it is possible to administer cyclosporin systemically via the skin.

The lack of suitable transport systems permitting the effect of a pharmacological agent, in particular of an immunomodulating agent, to be confined to the areas of the skin to be treated in each case is also shown by the fact that three important skin disorders which can be treated by modulation of the immune system, i.e. active psoriasis, atopic dermatitis and alopecia areata, are still treated with systemically administered cyclosporin A (Iconomidou et al. Dermatol. 199: 144-148, 1999; Naeyaert et al. Dermatol. 198: 145-152, 1999 and Ferrando and Grimalt, Dermatol. 199: 67-69, 1999).

Since disorders resembling the hair loss of human alopecia areata have been described in a number of animals, this disorder is suitable for use as model system to identify suitable transport systems. One form of reversible hair loss which corresponds clinically and histopathologically to human alopecia areata has been described, for example, for DEBR rats (Sundberg et al., J. Invest. Dermatol. 104: 32-33, 1995). DEBR rats develop the symptoms of the disorder about 6 months after birth, with the disorder having affected 20% of the animals in some rat colonies after 18 months.

### BRIEF SUMMARY OF THE INVENTION

An invasome comprising:

a) a lipid mixture comprising one or more lipids and one or more lysophosphatides, where the proportion of lysophosphatides in the lipid mixture is in the range from 0.1 to 40% by weight; and

b) at least one pharmacological agent.

The invasome of the present invention, where the lipids are selected from the group consisting of neutral lipids, anionic lipids and a mixture of neutral and anionic lipids.

The invasome according to claim 2, where the proportion of neutral or anionic lipids or a mixture of neutral and anionic lipids in the lipid mixture is in the range from 40 to 99.9% by weight.

The invasome of the present invention, where the invasome comprises the lipid mixture and the pharmacological agent in a ratio of from 1:1 to 1000:1, preferably from 2:1 to 100:1, by weight.

The invasome of the present invention, where the invasome has a diameter of from 30 to 400 nm.

The invasome of the present invention, where the lipid mixture is obtained from sources selected from the group of soybeans, cotton seeds, coconut kernel, peanut, safflower seeds, sesame seeds, sunflower seeds, linseeds, oilseed rape, wheatgerms, olives, whale fat, stratum corneum lipid, neatsfoot oil and egg.

The invasome of the present invention, where the proportion of neutral lipids in the lipid mixture is in the range from 75 to 95% by weight.

The invasome of the present invention, where the neutral lipids are selected from the group consisting of glycerophospholipids, in particular phosphatidylcholines, steroids, glycerophosphonolipids, glycerophosphinolipids and sphingolipids.

The invasome of the present invention, where the lysophosphatides are selected from the group consisting of lysophosphatidylcholines, lysophosphatidylethanol-amines, lysophosphatidylinositol, monolysocardiolipin, dilysocardiolipin and lysophosphatidylserines.

The invasome of the present invention, where the invasome comprises one or more terpenes.

In one embodiment of the invasome of the present invention, the terpene is selected from the group consisting of cineol, citral, limonene, in particular D-limonene, menthane, terpinene, terpinolene, menthol, in particular 1-menthol, carveol, in particular 1-carveol, menthone, carvone, pinene, in particular β-pinene, carene, in particular 3-carene, terpineol, terpinen-4-ol, pulegone, piperitone, cyclohexane oxide, limonene oxide, pinene oxide, cyclopentene oxide, ascaridol, 7-oxybicyclo[2.2.1]heptane, cymene, camphene, citronellol, geraniol, nerol, linalool, bomeol, thujol, sabinol, myrtenol, thymol, verbenol, fenchol, piperitol, perillaaldehyde, phellandral, citronellal, myrtenal, piperitone, thujone, umbellulone, verbenone, chrysanthenone, fenchone, camphor, quinone, menthofuran, linalool oxide, rose oxide and qinghaosu.

The invasome of the present invention, where the pharmacological agent is selected from the group consisting of an immunosuppressant, an immunostimulant, an antiallergic, an antibiotic, an antiinfective, a cytostatic, a cytotoxic agent, a mitogen, a chemokine, a cytokine, a dermatic and a physiological or pharmacological inhibitor of a mitogen, of a chemokine or of a cytokine.

In one embodiment of the invasome of the present invention, the immunosuppressant is selected from the group consisting of a glucocorticoid, in particular beclomethasone, betamethasone, clocortolone, cloprednol, cortisone, dexamethasone, fludrocortisone, fludroxycortide, flumetasone, fluocinolone acetonide, fluocinonide, fluocortolone, fluorometholone, fluprednidene acetate, hydrocortisone, paramethasone, prednisolone, prednisone, prednylidene, pregnenolone, triamcinolone or triamcinolone acetonide, a cyclosporin, in particular cyclosporin A, mycophenolate mofetil, tacrolimus, rapamycin, FK 506, cycloheximide-N-(ethyl ethanoate), azathioprine, ganciclovir, an anti-lymphocyte globulin, ascomycin, myriocin, pharmacological inhibitors of MAP kinases and methotrexate.

The invasome of the present invention, where the pharmacological agent is selected from the group consisting of a nucleic acid, a protein, a peptide, a sugar and a lipid.

In one embodiment of the invasome of the present invention, the nucleic acid is selected from the group consisting of an antisense oligonucleotide, an antisense RNA, an RNAi, an siRNA and an oligonucleotide which forms a triple helix.

A method for preparing an invasome of the present invention, characterized in that the lipid mixture is mixed with at least one pharmacological agent.

A medicament comprising an invasome of the present invention and suitable excipients and additives.

A method for using an invasome of the present invention for the therapy of a skin disorder.

In one embodiment of the method of the present invention, the skin disorder can be treated by modulation of the immune system.

In one embodiment of the method of the present invention, the skin disorder is selected from the group consisting of alopecia areata (all clinical forms), psoriasis vulgaris (all clinical forms), atopic dermatitis, atopic eczema, neurodermatitis, polymorphic light eruption, erythema solaris, allergic and irritative contact eczema, drug rash and graft versus host disease.

A method for using an invasome of the present invention for the therapy of a disorder which can be treated by modulation of the immune system, in particular for a prophylactic and/or therapeutic vaccination.

In one embodiment of the method of the present invention, the disorder is selected from the group consisting of an oncosis, a hyperplasia, a proliferative disorder, an arthritis, a viral disease, a bacterial and/or parasitic infection.

A method for using an invasome comprising a lipid mixture comprising one or more lipids and one or more lysophosphatides, where the proportion of lysophosphatides in the lipid mixture is in the range from 0.1 to 40% by weight, as adjuvant and/or carrier system for antigens and/or other immunomodulating molecules in the treatment of a disorder which can be treated by modulation of the immune system.

### BRIEF DESCRIPTION OF THE FIGURES AND TABLES

FIG. 1: Size of invasomes prepared under identical conditions as a function of the amount of terpene added.

FIG. 2: Anisotropy of invasomes prepared under identical conditions as a function of the amount of terpene added.

FIG. 3: Accumulation of cyclosporin A in the stratum corneum of the skin 6 h after nonocclusive treatment with cyclosporin A-containing invasomes.

FIG. 4: Profile of the distribution of ³H-cyclosporin A in abdominal human skin 6 hours after nonocclusive treatment with cyclosporin A-containing liposomes or invasomes (30 μl/cm²). The applied invasome mixture had a lipid content of 10% w/w.

FIG. 5: FACS analysis of suspensions of single cells from epidermis onto which FITC-containing empty invasomes have been applied topically beforehand.

FIG. 6: Epidermal sheet after topical application of empty invasomes. The dendritic cells are revealed dark by immunohistochemistry using the peroxidase technique.

Table 1: Hair growth after treatment with invasomes containing cyclosporin A or cyclosporin A and terpenes in the DEBR rat model. I: invasomes with terpenes (D-limonene, citral and cineol); F: invasomes without terpenes; C: control. Rats with natural hair growth were chosen as control group. The hair growth was assessed on the basis of the following arbitrary scale: −=large regions of the skin now completely free of hair; +=areas of the skin with very sparse hair growth; ++=areas of the skin with moderate hair growth; +++=apparently normal density of hair.

Table 2: The amount of cyclosporin A accumulated in the individual layers of the skin on use of various liposomes or invasomes is indicated as % of the amount of cyclosporin A applied. The total amount of cyclosporin determined in and on the skin is indicated in the last line. The applied invasome mixture had a lipid content of 10% w/w.

Table 3: The amounts of cyclosporin A accumulated in the organs liver, spleen, kidneys 48 h after use of cyclosporin A-containing invasomes were determined in three rats.

Table 4: Number of CD86-positive cells in the microscopic field of view on topical treatment with empty invasomes and with FITC-loaded invasomes.

### DETAILED DESCRIPTION OF THE INVENTION

Within the framework of the present invention, a cyclosporin A-loaded liposome form, called invasomes, has been found and could be used to treat successfully the partial hair loss in DEBR rats without the use of invasomes simultaneously leading to a measurable increase in the amount of cyclosporin A in the serum.

The present invention therefore relates to an invasome which comprises a lipid mixture comprising one or more lipids and one or more lysophosphatides, where the proportion of lysophosphatides in the lipid mixture is in the range from about 0.1 to about 40% by weight, and which comprises at least one pharmacological agent, preferably an immunosuppressant.

The term “invasome” refers to a unilamellar, bilamellar, oligolamellar (3, 4, 5, 6, 7, 8, 9 or 10 lamellae) or multilamellar (more than 10 lamellae) lipid-containing vesicle which transports only a small amount of the particular pharmacological agent present, in particular an immunomodulating agent, transdernally, so that the pharmacological agent, in particular an immunomodulating agent, reaches only a low concentration in the patient's serum and therefore can display only a small, or no, systemic effect. On use of the invasomes of the invention in therapeutically effective amounts it is possible to detect after the application, in particular after about 2 hours, about 4 hours, about 6 hours, about 9 hours, about 14 hours, about 24 hours, about 35 hours or after about 48 hours, not more than about 50 ng/ml, in particular not more than about 10 ng/ml, particularly preferably not more than about 7 ng/ml and most preferably not more than about 4 ng/ml of the particular pharmacological agent, in particular the immunomodulating agent, in the patient's serum. The serum concentration of the pharmacological agent, in particular an immunomodulating agent, about 2 hours after application is preferably below the limit of detection of the particular agent in the serum. Suitable detection methods depend on the pharmacological agent used, in particular immunomodulating agent, for example HPLC, ELISA, RT-PCR, scintillation or use of radiolabeled pharmacological agents, or gas chromatography. A therapeutically effective amount of the invasomes of the invention is, for example for invasomes which contain 0.5% cyclosporin A based on the total weight of the invasome mixture, from 20 to 80 μl of invasomes/cm² of treated skin.

The lamellar structure of the invasome of the invention can also be formed by an odd number of lamellae, such as, for example, 1, 3, 5, 7, 9 or 11 lamellae, in which case one side forms the hydrophobic interior of the vesicle and the other side forms the hydrophilic exterior. This type of vesicle is particularly suitable for uptake of, for example, hydrophobic pharmacological agents, immunosuppressants.

The invasomes of the invention comprise the lipid mixture and the pharmacological agent(s), in particular the immunomodulating agent(s), in a ratio in the range from about 1:1 to about 1000:1 [lipid mixture:pharmacological agent(s)], preferably in the range from about 2:1 to about 100:1, more preferably in the range from about 5:1 to about 50:1, even more preferably in the range from about 15:1 to about 30:1 and most preferably in the range from about 18:1 to about 22:1, by weight.

The lipid mixture present in the invasome is derived from plant sources, animal sources and/or is of completely synthetic origin. The lipids are preferably neutral and/or anionic lipids. The proportion of neutral and anionic lipids in a lipid mixture derived from plant sources and/or animal sources can be increased by further purification methods directed at the enrichment of neutral and/or anionic lipids. The proportion of neutral or anionic lipids or a mixture of the two lipid forms is preferably in the range from about 40 to about 99.9% by weight of the total amount of the lipid mixture. Preference is therefore given to lipid mixtures from plant or animal sources which naturally contain neutral and/or anionic lipids in the stated range. Methods for obtaining fats and oils and for enrichment of individual components are known in the art and are described, for example, in Ullmanns Enzyklopädie der technischen Chemie, volume 11, pages 455-523, 1976. The term lipid mixture also encompasses mixtures which are derived from lipids from various sources, for example different plants, especially if the mixture contains neutral and/or anionic lipids in the range from about 40 to about 99.9% by weight. In a particularly preferred embodiment, the lipid mixture contains neutral lipids in the range from about 40 to about 99.9% by weight.

A lipid for the purpose of the present invention is any substance which has fatty or fat-like properties and where this substance has an extensive apolar portion and a polar, water-soluble portion. These include, for example, neutral, anionic or cationic lipids, but not lysophosphatides.

A lipid mixture for the purpose of the present invention is any mixture of the aforementioned lipids which additionally comprises one or more lysophosphatides.

A neutral lipid for the purpose of the present invention is a lipid which comprises one or more positive and negative charge carriers, with the same number of positive and negative charge carriers being present in the polar portion in each case. For this reason, the overall charge of invasomes which comprise neutral lipids is neutral in the range from about pH 6.0 to about 8.0, preferably in the range from about pH 6.5 to about 7.5 and particularly preferably at about pH 7.0, depending on the neutral lipid(s).

An anionic lipid for the purpose of the present invention is a lipid which contains no or a plurality of positive and one or more negative charge carriers, with the number of negative charge carriers being higher than the number of positive charge carriers in the polar portion. For this reason, the overall charge of invasomes which comprise anionic lipids is negative in the range from about pH 6.0 to about 8.0, preferably in the range from about pH 6.5 to about 7.5 and particularly preferably at about pH 7.0, depending on the anionic lipid(s).

If the lipid mixture comprises a mixture of neutral and anionic lipids, the invasomes have a negative overall charge.

The invasomes of the invention are further composed of one or more lysophosphatides, where the proportion of lysophosphatides in the mixture is in the range from about 0.1 to about 40% by weight, preferably from about 1 to about 25% by weight, even more preferably from about 4 to about 15% by weight and most preferably from about 10 to about 15% by weight.

Examples of preferred lysophosphatides which can be used for the purpose of the present invention are lysophosphatidylcholine (lysolecithin), lysophosphatidyl-ethanolamine, lysophosphatidylinsolitol, lysophosphatidylserine, monolysocardiolipin or dilysocardiolipin. A particularly preferred lysophosphatide is lysolecithin. Naturally obtained or purified lipid mixtures in some cases contain lysophosphatides within the stated weight ranges. Otherwise, it is possible to add natural or synthetic lipids, preferably neutral and/or anionic lipids, natural or synthetic lyosphosphatides in the stated weight ranges.

An invasome of the invention has a size between 20 and 1000 nm. The size distribution of the invasomes depends on the method of preparation. Thus, sonication of the lipid mixtures leads to small vesicles, whereas “vortexing” leads to larger vesicles. Vesicles within a particular size class can be selected by methods known in the art, such as microfiltration or centrifugation.

The invasomes of the invention have, in a preferred embodiment, a diameter in the range from about 50 to about 400 nm, in particular in the range from about 60 to about 200 nm and particularly preferably in the range from about 80 to about 150 nm. The size of the vesicles influences the ability of the vesicles to permeate, with small vesicles in the range from about 50 to about 200 nm displaying a better ability to penetrate through the stratum corneum than larger vesicles with a diameter of, for example, from about 600 to about 800 nm.

Preferred sources for obtaining the lipid mixture which can be used to prepare an invasome of the invention are lipid mixtures obtained from oil seeds, in particular from soybeans, cotton seeds, coconut kernel, peanut, safflower seeds, sesame seeds, sunflower seeds, linseeds, oilseed rape, wheatgerms, olives or animal fats, in particular whale fat, stratum corneum lipid, neatsfoot oil and/or egg. Soybeans are a particularly preferred source of lipid mixtures. Enrichment of the neutral lipids, anionic lipids and/or lysophosphatides in the lipid mixtures which can be obtained from the starting materials is possible for example by chromatographic methods such as HPLC and other methods known in the art (see Ullmann, 1976, Supra).

In a preferred embodiment of the invasome of the invention, the proportion by weight of the neutral, anionic lipid or mixture of the two lipid forms, in particular of the neutral lipid, in the lipid mixture is in the range from about 50 to about 98% by weight, preferably in the range from about 60 to about 95% by weight, even more preferably in the range from about 75 to about 95% by weight and most preferably about 90% by weight. An increase in the proportion of the neutral, anionic or of a mixture of the two lipid forms above about 40% in the lipid mixture leads to an improvement in the permeation properties, but these deteriorate again when the proportion of neutral or anionic lipid is increased further to above 95% by weight in the lipid mixture. Thus, a pure neutral lipid recovers the ability to penetrate through the stratum corneum only through addition of surface-active substances (see Guo et al., 2000 Supra and WO 92/03122), such as, for example, surfactants, but, on the other hand, this also leads to penetration through the other layers of the skin and finally to systemic release of the particular agent.

In the further preferred embodiment of the invasome of the invention, the neutral lipid is a glycerophospholipid, in particular a phosphatidylcholine, a steroid, a glycerophosphonolipid, a glycerophosphinolipid or a sphingolipid. However, particular preference is given in this connection to a glycerophospholipid, in particular a phosphatidylcholine. Suitable phosphatidylcholine-containing lipid mixtures are commercially available for example with a proportion of about 80% by weight of the complete lipid mixture. Phospholipon 80 (Nat 8539; Nattermann Phospholipid GmbH) is such a lipid mixture, which is also obtainable as ethanolic solution and has the following composition: 73.0-79.0% by weight (3-sn-phosphatidyl)choline, 6% by weight (3-sn-lysophosphatidyl)choline, 7% by weight cephalin and 7% by weight phosphatidic acid.

In a further preferred embodiment of the invasome of the invention, the anionic lipid is a phosphatidylserine, a phosphatidylglycerol, a phosphatidic acid, phosphatidylinositol and/or a cholesterol glutarate.

The invention further relates to an invasome which additionally comprises one or more terpenes. Terpenes for the purpose of the present invention may encompass products of the polymerization of the hydrocarbon isoprene. According to the number of isoprene residues, a distinction is made between monoterpenes (C₁₀), sesquiterpenes (C₁₅), diterpenes (C₂₀), sesterpenes (C₂₅), triterpenes (C₃₀), tetraterpenes (C₄₀) and polyterpenes. The acyclic hydrocarbons formed from the basic isoprene units can be converted into a large number of compounds for example by subsequent substitutions, oxidation, cyclizations and rearrangement. Terpenes for the purpose of the present invention also means the derived alcohols, ketones, aldehydes and esters.

Terpenes are added to the lipid mixture in the range from about 0.1 to about 200% by weight based on the amount of the lipid mixture. Preferably, however, in the range from about 5 to about 100% by weight, more preferably in the range from about 10 to about 50% by weight and most preferably in the range from about 15 to about 30% by weight. If the added amount of terpene is too high, the terpenes suppress formation of the invasomes. The amount of terpene which prevents invasome formation in each case depends on the lipid mixture used and on the pharmacological agent used. It is easy to test whether the chosen amount of terpene prevents invasome formation by one of the methods described hereinafter for preparing invasomes of the invention. Measurements of the anisotropy of invasomes have shown that invasomes containing terpenes have reduced flexibility compared with non-terpene-containing invasomes.

In a preferred embodiment, the invasome of the invention comprises one or more of the following terpenes: cineol, citral, limonene, in particular D-limonene, menthane, terpinene, terpinolene, menthol, in particular 1-menthol, carveol, in particular 1-carveol, menthone, carvone, pinene, in particular β-pinene, carene, in particular 3-carene, terpineol, terpinen-4-ol, pulegone, piperitone, cyclohexane oxide, limonene oxide, pinene oxide, cyclopentene oxide, ascaridol, 7-oxybicyclo[2.2.1]heptane, cymene, camphene, citronellol, geraniol, nerol, linalool, bomeol, thujol, sabinol, myrtenol, thymol, verbenol, fenchol, piperitol, perillaaldehyde, phellandral, citronellal, myrtenal, piperitone, thujone, umbellulone, verbenone, chrysanthenone, fenchone, camphor, quinone, menthofuran, linalool oxide, rose oxide and qinghaosu, including the various structural and configurational isomers of the aforementioned terpenes.

The invasome may comprise individual terpenes or mixtures of terpenes. A particularly preferred invasome comprises a mixture of D-limonene, cineol and citral. Preferred mixing ratios for these terpenes are in the range from about 2:49:49 to about 1:1:1, preferably in the range from about 4:48:48 to about 1:3:3 and particularly preferably in the region of about 10:45:45 (D-limonene:cineol:citral).

Pharmacological agents for the purpose of the present invention are all substances which have a therapeutic effect, but especially substances which are employed in the therapy of skin disorders. Preferred pharmacological agents are therefore immunosuppressants, immunostimulants, antibiotics, antiinfectives, antiallergics, cytostatics, cytotoxic agents, mitogens, chemokines, cytokines, dermatics and/or physiological or pharmacological inhibitors of mitogens, chemokines, cytokines.

Particularly preferred pharmacological agents are immunosuppressants and immunostimulants.

The term “immunosuppressant” encompasses all substances which influence the function of cells which are involved directly or indirectly in mediation of the immune response, and where the influence leads to a diminution of the immune response. These cells include, for example, macrophages, dendritic cells, Langerhans cells, indeterminate cells, but also cells which do not themselves belong to the immune system but are involved in immune disorders of the skin, such as fibroblasts, keratinocytes and melanocytes. The strength of the immune response can be determined for example through the amount of cytokines produced (such as interferon-gamma), detection of activation markers on dendritic cells (such as MHCII or CD86) or the number of activated CD8-positive T cells in the skin.

In a preferred embodiment of the invasome of the invention, the immunosuppressant is a glucocorticoid, in particular beclomethasone, betamethasone, clocortolone, cloprednol, cortisone, dexamethasone, fludrocortisone, fludroxycortide, flumetasone, fluocinolone acetonide, fluocinonide, fluocortolone, fluorometholone, fluprednidene acetate, hydrocortisone, paramethasone, prednisolone, prednisone, prednylidene, pregnenolone, triamcinolone or triamcinolone acetonide, a cyclosporin, in particular cyclosporin A, mycophenolate mofetil, tacrolimus, rapamycin, FK 506, cycloheximide-N-(ethyl ethanoate), azathioprine, ganciclovir, an anti-lymphocyte globulin, ascomycin, myriocin, a pharmacological inhibitor of MAP kinases (especially a p38 inhibitor such as VX-745) and/or methotrexate. A particularly preferred invasome of the present invention comprises cyclosporin A as immuno-suppressant.

The term “immunostimulant” encompasses all substances which influence the function of cells which are involved directly or indirectly in mediation of the immune response, and where the influence leads to an immune response. These cells include, for example, macrophages, Langerhans cells and other dendritic cells, lymphocytes, indeterminate cells, but also cells which do not themselves belong to the immune system but are involved in immune disorders of the skin, such as fibroblasts, keratinocytes and melanocytes, but especially Langerhans cells. The strength of the immune response can be determined for example through the amount of cytokines produced (such as interferon-gamma), detection of activation markers on dendritic cells (such as MHCII or CD86) or the number of activated CD8-positive T cells in the skin. Immunostimulants for the purpose of the present invention are, in particular, plant immunostimulants which are obtained, for example, from Echinacea pallida or Echinacea purpurea, cytokines such as, for example, interleukins, interferons and colony-stimulating factors, and bacterial constituents or molecules which mimic the latter [such as bacterial DNA and unmethylated oligodeoxynucleotides with CpG sequences, and constituents of the bacterial cell wall or coat, especially the lipopolysaccharides and molecules derived therefrom, such as monophosphoryl-lipid A, muramyldipeptide (N-acetylmuramyl-L-alanyl-D-isoglutamine), and/or PamCys3, and other molecules such as tetanus toxoid, poly-L-arginine or MHCII peptides].

The term “antibiotics” encompasses, for example, penicillins, cephalosporins, tetracyclines, aminoglycosides, macrolide antibiotics, lincosamides, gyrase inhibitors, sulfonamides, trimethoprim, polypeptide antibiotics, nitroimidazole derivatives, amphenicol, especially actinomycin, alamethicin, alexidine, 6-aminopenicillanic acid, amoxicillin, amphotericin, ampicillin, anisomycin, antiamoebin, antimycin, aphidicolin, azidamfenicol, azidocillin, bacitracin, beclomethasone, benzathine, benzylpenicillin, bleomycin, bleomycin sulfate, calcium ionophore A23187, capreomycin, carbenicillin, cefacetrile, cefaclor, cefamandole nafate, cefazolin, cefalexin, cefaloglycin, cefaloridine, cefalotin, cefapirin, cefazolin, cefoperazone, ceftriaxone, cefuroxime, cephalexin, cephaloglycin, cephalothin, cephapirin, cerulenin, chloramphenicol, chlortetracycline, chloramphenicol diacetate, ciclacillin, clindamycin, chlormadinone acetate, chlorpheniramine, chromomycin A3, cinnarizine, ciprofloxacin, clotrimazole, cloxacillin, colistine methanesulfonate, cycloserine, deacetylanisomycin, demeclocycline, 4,4′-diaminodiphenyl sulfone, diaveridine, dicloxacillin, dihydrostreptomycin, dipyridamole, doxorubicin, doxycycline, epicillin, erythromycin, erythromycin stolate, erythromycin ethyl succinate, erythromycin stearate, ethambutol, flucloxacillin, fluocinolone acetonide, 5-fluorocytosine, filipin, formycin, fumaramidomycin, furaltadone, fusidic acid, geneticin, gentamycin, gentamycin sulfate, gliotoxin, gramicidin, griseofulvin, helvolic acid, hemolysin, hetacillin, kasugamycin, kanamycin (A), lasalocid, lincomycin, magnesidin, melphalan, metacycline, meticillin, mevinolin, micamycin, mithramycin, mithramycin A, mithramycin complex, mitomycin, minocycline, mycophenolic acid, myxothiazole, natamycin, nafcillin, neomycin, neomycin sulfate, 5-nitro-2-furaldehyde semicarbazone, novobiocin, nystatin, oleandomycin, oleandomycin phosphate, oxacihin, oxytetracycline, paromomycin, penicillin, pecilocin, pheneticillin, phenoxymethylpenicillin, phenyl aminosalicylate, phleomycin, pivampicillin, polymyxin B, propicillin, puromycin, puromycin aminonucleoside, puromycin aminonucleoside 5′-monophosphate, pyridinol carbamate, rolitetracycline, rifampicin, rifamycin B, rifamycin SV, spectinomycin, spiramycin, streptomycin, streptomycin sulfate, sulfabenzamide, sulfadimethoxine, sulfamethizole, sulfamethoxazole, tetracycline, thiamphenicol, tobramycin, troleandomycin, tunicarnycin, tunicamycin A1 homolog, tunicamycin A2 homolog, valinomycin, vancomycin, vinomycin A1, virginiamycin M1, viomycin and/or xylostasin.

The term “antiinfectives” encompasses, for example, antimycotics, agents with antiparasitic effect and virustatics, in particular amphotericin, vifonazole, buclosamide, quinoline sulfate, chlormidazole, chlorphenesin, chlorquinaldol, clodantoin, cloxiquine, cyclopirox olamine, dequalinium chloride, dimazole, fenticlor, flucytosine, griseofulvin, ketoconazole, miconazole, natamycin, sulbentine, tioconazole, toinaftate, antiretroviral agents and/or herpes remedies.

The term “antiallergics” encompasses for example substances from the class globulins, corticoids or antihistamines, in particular beclomethasone derivatives, betamethasone cortisone derivatives, dexamethasone derivatives, bamipine acetate, buclizine, clemastine, clemizole, cromoglicic acid, cyproheptadine, diflucortolone valerate, dimetotiazine, diphenhydramine, diphenylpyraline, ephedrine, fluocinolone, histapyrrodine, isothipendyl, methdilazine, oxomemazine, paramethasone, prednylidene, theophylline, and/or tolpropamine tritoqualine.

The term “cytostatics” and “cytotoxic agents” encompass for example alkylating substances, antibiotics, platinum compounds, hormones and antihormones, interferons and inhibitors of cell cycle-dependent protein kinases (CDKs), in particular acediasulfone, scriflavinium chloride, ambazone, dapsone, dibrompropamidine, furazolidone, hydroxymethylnitrofurantoin, idoxuridine, mafenide sulfate olamide, mepacrine, metronidazole, nalidixic acid, nifuratel, nifuroxazide, nifuralazine, nifurtimox, ninorazole, nitrofurantoin, oxolinic acid, pentamidine, phenazopyridine, phthalylsulfathiazole, pyrimethamine, salazosulfapyridine, sulfacarbamide, sulfacetamide, sulfachlopyridazine, sulfadiazine, sulfadicramide, sulfadimethoxine, sulfaethidole, sulfafurazole, sulfaguanidine, sulfaguanole, sulfamethizole, sulfamethoxazole, co-trimoxazole, sulfamethoxydiazine, sulfamethoxypyridazine, sulfamoxole, sulfanilamide, sulfaperin, sulfaphenazole, sulfathiazole, sulfisomidine, tinidazole, trimethoprim, aclarubicin, azathioprine, bleomycin, busulfan, calcium folinate, carboplatin, carmustine, chlorambucil, cis-platin, cyclophosphamide, cytarabine, daunorubicin, epirubicin, fluorouracil, fosfestrol, hydroxycarbamide, ifosfamide, lomustine, melphalan, mercaptopurine, methotrexate, mitomycin C, mitopodozide, mithramycin, nimustine, pipobroman, prednimustine, procarbazine, testolactone, treosulfan, thiotepa, thioguanine, triaziquone, trofosfamide, vincristine, vindesine, vinblastine, zorubicin, flavopiridol, oleomucin and/or preussin.

The terms “mitogens”, “chemokines” and “cytokines” encompass, for example, interferon-alpha, interferon-beta, interferon-gamma, interleukin-1, interleukin-2, interleukin-7, interleukin-10, interleukin-12, interleukin-18, GM-CSF, MIP-1-alpha/beta, RANTES, EGF, basic or acidic FGF, PDGF, IGF, VEGF, TGF-beta and/or TNF-alpha.

The term “dermatics” encompasses, for example, shale oil sulfonates, tar and tar derivatives, astringents, antihidrotics, acne remedies, antipsoriatics, antiseborrheic agents and/or enzyme preparations for the treatment of skin defects.

In one embodiment, the pharmacological agent is a nucleic acid, a protein, a peptide, a sugar and/or a lipid, for example bacterial DNA, unmethylated oligodeoxynucleotides with CpG sequences, lipopolysaccharides, lipid A, muramyldipeptide and/or PAMCys3.

In a preferred embodiment, the nucleic acid is an antisense oligonucleotide (Agrawal & Zhao, Curr. Opin. Chem. Biol. 2: 519-28, 1998), an antisense RNA (Weintraub, Sci. Am. 262: 40-6, 1990), an RNAi (Carthew, Curr. Opin. Cell. Biol. 13: 244-8, 2001), an siRNA (Elbashir et al. Nature 411: 494-6, 2001) or an oligonucleotide forming a triple helix (Casey & Glazer, Prog. Nucleic Acid Res. Mol. Biol. 67: 163-92, 2001).

In a further embodiment, the nucleic acid codes for at least one pharmacologically active peptide and/or protein, in particular for an immunomodulating peptide or protein. In this connection, peptide means an amino acid chain with a length of from 2 to 50 amino acids and protein means an amino acid chain which is longer than 50 amino acids. In this case, the nucleic acids present in the invasome are introduced into skin cells; such an introduction is also referred as transfection.

Suitable nucleic acids code, for example, for interferon-alpha, interferon-beta, interferon-gamma, interleukin-1, interleukin-2, interleukin-7, interleukin-10, interleukin-12, interleukin-18, GM-CSF, MIP-1-alpha/beta, RANTES, TGF-beta, TNF-alpha and/or CTLA4-Ig (see, for example, Abrams et al. J. Clin Invest 103: 1243-52, 1999). The invention also emcompasses nucleic acids which code for the aforementioned peptides or proteins whose amino acid sequence has, however, additions, substitutions, deletions or mutations of individual amino acids or groups of amino acids. Preference is given in this connection to so-called conservative mutations such as, for example, exchange of a glutamic acid residue for an aspartic acid residue, deletion or substitution of regions of the protein which are not responsible for the pharmacological, in particular immunomodulating, effect. The nucleic acids may be in the form, for example, of single-stranded or double-stranded DNA or of RNA, but preferably of double-stranded DNA:

The nucleic acid may be part of a vector, preferred vectors being those leading on transfection of a cell to sustained expression of the pharmacological agent, in particular immunomodulating agent, such as, for example, episomally or extrachromosomally replicating vectors.

The nucleic acid may additionally comprise regulatory elements 5′ or 3′ from the open reading frame coding for the pharmacological agent, in particular the immunomodulating peptide or protein. Examples of regulatory elements suitable for expression are those permitting constitutive, regulatable, tissue-specific, cell cycle-specific or metabolically specific expression in eukaryotic cells. Regulatory elements according to the present invention encompass promoter, activator, enhancer, silencer and/or repressor sequences.

Examples of suitable regulatory elements which make constitutive expression possible in eukaryotes are promoters recognized by RNA polymerase III or viral promoters, CMV enhancer, CMV promoter, SV40 promoter or LTR promoters, for example of MMTV (mouse mammary tumour virus; Lee et al. (1981) Nature 214, 228-232) and viral promoter and activator sequences derived, for example, from HBV, HCV, HSV, HPV, EBV, HTLV or HIV.

Examples of regulatory elements which make inducible expression possible in eukaryotes are the tetracycline operator in combination with an appropriate repressor (Gossen et al. Curr. Opin. Biotechnol. 5, 516-20, 1994).

Examples of regulatory elements which make tissue-specific expression possible in eukaryotes are promoters or activator sequences from promoters or enhancers of genes which code for proteins which are expressed only in particular cell types, preferably cells of the immune system. A large number of immune cell-specific regulatory elements has been described in the prior art, and all of them are suitable for regulating expression of the immunomodulating agent, but particular preference is given to promoters active in T cells or promoter fragments active in T cells, such as, for example, promoters or promoter fragments of the following genes: CD4, CD8 (described, for example, in Ellmeier et al. Annu. Rev. Immunol. 17: 523-54, 1999), IL-3, IL-4, IL-5, IL-13, GM-CSF (described, for example, in De Boer et al. Int. J. Biochem. Cell Biol. 31: 1221-36, 1999) or synthetic promoters with NF-AT binding sites (described, for example, in Hooijberg et al.

Blood 96: 459-466, 2000). Particularly suitable promoters are active only in activated T cells and thus permit the effect of the immunomodulating agent(s) to be confined to the activated T cells themselves and their immediate environment.

In a preferred embodiment, the invasome of the invention is able to transport therapeutically effective amounts of at least one pharmacological agent, in particular an immunomodulating agent, through the stratum corneum of the skin. The transition from the stratum corneum to deeper layers of skin, such as, for example, the stratum granulosum, the stratum spinosum and the stratum basale, can be determined histologically. For example, the transition from flat, anuclear and cornified cells to flattened granular cells is characteristic of the end of the stratum corneum and the start of the stratum granulosum. The methods and criteria for histological determination of these transitions are known to the skilled worker. Distribution of the pharmacological agent, in particular immunosuppressant, in the skin can be determined for example using an adhesive strip pull-off method (Michel et al., Int. J. Pharm 84: 93-105, 1992). This entails use of the invasomes of the invention being followed by removal of the skin layer by layer using adhesive strips, and then determining the amount of the pharmacological agent, in particular an immunomodulating agent, as a function of the distance from the skin surface. Determination of the therapeutic effect can take place for example for immunosuppressants as in example 2, in which invasomes which contain an immunomodulating agent or invasomes which contain no immunomodulating agent are applied in each case to two adjoining areas of the skin of a patient or of an animal suffering from a skin disorder which can be treated by modulation of the immune system of the skin. The therapeutic effect can be determined via a regression in clinical parameters, change in the amount of cytokines expressed in the skin and/or change in the number of immune cells or activated immune cells in the skin. On use of an immunosuppressant it is possible to determine the therapeutic effect for example via a reduction in reddening, swelling and/or warmth or by the reduction in the amount of cytokines expressed in the skin and/or the reduction in the number of immune cells or activated immune cells in the skin. When an immunosuppressant is used as pharmacological agent there is a therapeutic effect if expression of the respective investigated cytokine in the skin which has been treated with invasomes containing an immunosuppressant is reduced, compared with skin treated with invasomes without immunosuppressant, by at least about 2-fold, preferably by at least about 5-fold, more preferably by at least about 10-fold and most preferably by at least about 20-fold; or if the amount of immune cells, in particular of activated immune cells, in the skin which has been treated with invasomes containing immunosuppressant is reduced, compared with skin treated with invasomes without immunosuppressant, by at least about 30%, preferably by at least about 100%, more preferably by at least about 200% and most preferably by at least about 500%. When an immunostimulant is used as pharmacological agent there is a therapeutic effect if expression of the respective investigated cytokine in the skin which has been treated with invasomes containing an immunostimulant is increased, compared with skin treated with invasomes without immunostimulant, by at least about 2-fold, preferably by at least about 5-fold, more preferably by at least about 10-fold and most preferably by at least about 20-fold, or if the amount of immune cells, in particular of activated immune cells, in the skin which has been treated with invasomes containing immunostimulant is increased, compared with skin treated with invasomes without immunostimulant, by at least about 30%, preferably by at least about 100%, more preferably by at least about 200% and most preferably by at least about 500%. Suitable cytokines whose concentration can be investigated to determine the pharmaceutical effect encompass, for example, interferon-alpha, interferon-beta, interferon-gamma, interleukin-2, interleukin-7, interleukin-10, interleukin-12, interleukin-18, GM-CSF, TGF-beta and/or TNF-alpha. The number of immune cells in the various regions of the skin can be determined for example by immunohistological investigation of thin sections of the skin or by FACS analysis of single-cell suspensions of the skin which have previously been incubated with an immune cell-specific marker. Examples of suitable markers are anti-CD28, anti-CD3, anti-CD86 and/or anti-MHC II antibodies.

With the other pharmacological agents it is possible to assume a therapeutic effect if the respective symptoms of the disorder are markedly improved within a period of 10-200 days after administration. There is a marked improvement if the parameters of the disorder which can be determined by a clinician, such as, for example, the size of a lesion, the severity of the reddening, the growth of the tumor cells, declines by at least about 30%, preferably by 50%, even more preferably by about 80% and most preferably by about 100%.

The immune system of the skin is formed on the one hand by the cells already present in the skin, such as, for example, dendritic cells, but also by the immune cells infiltrating into the skin in each case of a disorder, infection and/or inflammatory processes, such as, for example, T cells, LangeFhans cells and macrophages. When the immune system of the skin is involved there is observed to be by comparison with healthy skin an increase in infiltration of immune cells into the skin or activation of said immune cells without an injury or infection of the skin in fact being present. It is possible to identify whether the immune system of the skin is involved in a skin disorder through the parameters of reddening, warmth and/or swelling of the skin which can be determined clinically. If these are found, a further criterion which is preferably used is the amount of cytokines expressed in the skin and/or the number of immune cells or activated immune cells in the skin.

In a preferred embodiment of the invasome of the invention, the invasome contains no surfactant because surfactants lead to an increase in the ability of liposomes to permeate, which lead to a complete penetration through the skin and thus to a systemic effect of the pharmacological agent. The term surfactants encompasses for the purpose of this invention nonionic surfactants such as, for example, Tween or Triton, zwitterionic surfactants such as, for example, CHAPS or CHAPSO, cationic or anionic surfactants such as, for example, lauryl sulfate or laurolyl, but not lysophosphatides such as, for example, lysophosphatidylcholine, lysophosphatidylethanolamine, lysophosphatidylinositol, monolysocardiolipin, dilysocardiolipin or lysophosphatidylserine.

The present invention further relates to a method for preparing an invasome, characterized in that the lipid mixture which comprises one or more lipids and one or more lysophosphatides, where the proportion of lysophosphatides in the lipid mixture is in the range from about 0.1 to about 40% by weight, and at least one pharmacological agent, in particular an immunomodulating agent, are mixed.

In a preferred embodiment of the method of the invention, the lipid mixture comprises neutral lipids, anionic lipids or a mixture of the two lipid forms, but especially neutral lipids, in a proportion in the range from about 40 to not more than about 99.9% by weight.

In one embodiment of the method of the invention, an alcoholic solution of the lipid mixture is mixed with at least one pharmacological agent, in particular an immunomodulating agent, for example by vortexing. Alcohols suitable for dissolving the lipid mixture are, for example, ethanol, n-propanol, isopropanol, n-butanol, isobutanol and/or n-pentanol, but also longer-chain alcohols or higher alcohols suitable for dissolving the particular lipid mixture. It is possible to add to the mixture where appropriate terpenes before or after the addition of at least one pharmacological agent, in particular an immunomodulating agent. The lipid mixture can be dissolved in the alcohol in a ratio in the range from about 10:1 to about 1:100 (alcohol:lipid mixture), preferably in the range from about 2:1 to about 1:20, more preferably in the range from about 1:1 to about 1:10, even more preferably from about 1:2 to about 1:8 and most preferably from about 1:3 to about 1:4, by weight.

The mixture (with or without terpene(s)) can then be sonicated with ultrasound. In a further step, buffer or else distilled water can be added with stirring to a mixture of lipid mixture and a pharmacological agent, in particular an immunomodulating agent, which comprises alcohol and/or terpenes where appropriate. A suitable buffer is any physiologically tolerated buffer, in particular phosphate buffer. The added buffer or the added water can in this case constitute in the range from about 5 to about 98% by weight, preferably from about 30 to about 95% by weight, particularly preferably from about 60 to about 90% by weight, more preferably from about 80 to about 88% by weight, of the complete mixture. This step results in a so-called coarse-grain suspension in which the invasomes of the invention are suspended. The invasomes prepared in this way are suspended in the buffer but may also comprise buffer or water.

These multilamellar vesicles can in a further step, to prepare invasomes of very particular size, for example be sonicated or extruded through polycarbonate membranes with a defined pore width such as, for example, 400, 200, 100 or 50 nm.

Another method known in the art for preparing liposomes which is also suitable for preparing the invasomes of the invention is the rotary evaporation method (Weiner et al., Antimicrob. Agents Chemother. 33: 1217-1221, 1989). In this method, the lipid mixture is dried on the bottom of a round-bottomed flask and then dispersed through addition of an aqueous solution which contains the pharmacological agents, in particular immunomodulating agents, and, where appropriate, terpenes, alcohols and/or buffers, with shaking. The hydration is followed by extrusion of the resulting multilamellar vesicles for example through polycarbonate membranes of defined pore size, or else sonication in order to obtain invasomes of a particular size.

In a preferred embodiment of the method of the invention, the lipid mixture used and the pharmacological agent, in particular immunomodulating agent, contains no surfactant. The term surfactants encompasses for the purpose of this invention nonionic surfactants such as, for example, Tween or Triton, zwitterionic surfactants such as, for example, CHAPS or CHAPSO, cationic or anionic surfactants such as, for example, lauryl sulfate or laurolyl. The term surfactant does not, however, include lysophosphatides such as, for example, lysophosphatidylcholine, lysophosphatidylethanolamine, lysophosphatidylinositol, monolysocardiolipin, dilysocardiolipin or lysophosphatidylserine.

The present invention also encompasses invasomes prepared by one of the aforementioned methods.

The present invention further relates to a medicament which comprises an invasome of the invention and suitable excipients and additives. Examples of such excipients and additives are physiological saline solutions, Ringer dextrose, dextrose, Ringer lactate, demineralized water, stabilizers, antioxidants, complexing agents, antimicrobial compounds, proteinase inhibitors, inert gases, gel formulations such as, for example, white petrolatum and/or paraffin. The medicament may also be prepared for topical application in the form of pressings, plasters, compresses, ointments or gels.

The present invention further relates to the use of an invasome of the invention for the therapy of a skin disorder. Skin disorders which can be treated with the invasomes of the invention encompass, for example, inflammations of the skin, autoimmune diseases of the skin, allergies, graft versus host reaction, mycoses, bacterial and viral infections, tumors and other proliferative disorders of the skin.

A preferred use of the invasomes of the invention is the therapy of skin disorders which can be treated by modulation of the immune system of the skin. The term therapy in this connection encompasses both curative or palliative treatment of pre-existing disorders and the prevention of disorders.

The immune system of the skin is formed on the one hand by the cells already present in the skin, such as, for example, dendritic cells, but also by the immune cells infiltrating into the skin in each case of a disorder, infection and/or inflammatory processes, such as, for example, T cells, Langerhans cells and macrophages. In the case of a disorder which can be treated by modulation of the immune system of the skin there is observed to be by comparison with healthy skin an increase in infiltration of immune cells into the skin or activation of said immune cells without an injury or infection of the skin in fact being present. It is possible to identify whether the immune system of the skin is involved in a skin disorder through the parameters of reddening, warmth and/or swelling of the skin which can be determined clinically. If these are found, a further criterion which is preferably used is the amount of cytokines expressed in the skin and/or the number of immune cells or activated immune cells in the skin.

Cytokines which show enhanced expression in skin disorders which can be treated by modulation of the immune system of the skin encompass, for example, interferon-γ, IL-2, IL-1β or IL-12. The amount of cytokines expressed in the skin can be determined at the nucleic acid level, for example by RT-PCR, RNAse protection assays or nuclear run ons, and at the protein level for example by Western blots or ELISA. The skilled worker is aware of other methods which allow the level of expression of cytokines in the skin to be determined. In this connection, the level of expression determined in each case in the diseased skin is compared with the level of expression in healthy skin. A skin disorder which can be treated by modulation of the immune system of the skin is present if the expression of the particular cytokine in the diseased skin is increased compared with healthy skin by at least about 2-fold, preferably by at least about 5-fold. In this case, an immunosuppressant is used as pharmacological agent.

A skin disorder which can be treated by modulation of the immune system of the skin is also present if expression of the particular cytokine in the diseased skin is reduced compared with healthy skin to less than 50%, preferably to less than 20%. In this case, an immunostimulant is used as pharmacological agent.

Immune cells which occur in larger amount in the skin in skin disorders which can be treated by modulation of the immune system of the skin encompass, for example, CD4⁺ cells, CD8⁺ cells, Langerhans cells and macrophages. The amount of the immune cells occurring in the skin can be determined for example by FACS analysis, histologically and/or immunohistologically. A skilled worker is aware of other methods which permit the amount of immune cells in the skin to be determined. In this connection, the amount of immune cells determined in each case, preferably the amount of activated immune cells, in the diseased skin is compared with the amount of immune cells, preferably the amount of activated immune cells, in healthy skin. Besides the criterion of cytokine expression, therefore, a further criterion, which may be present alone or at the same as the aforementioned criterion, is the amount of activated immune cells in the skin. A skin disorder which can be treated by modulation of the immune system of the skin is also present if the amount of immune cells, in particular of activated immune cells, in the diseased skin is increased compared with the healthy skin by at least about 50%, preferably by at least about 100%, more preferably by at least about 200% and most preferably by at least about 500%. An immunosuppressant is used as pharmacological agent in this case.

A skin disorder which can be treated by modulation of the immune system of the skin is also present if the amount of the immune cells, in particular of the activated immune cells, in the diseased skin is reduced compared with the healthy skin by at least about 50%, preferably by at least about 100%, more preferably by at least about 200% and most preferably by at least about 500%. An immunostimulant is used as pharmacological agent in this case.

A skin disorder which can be treated by modulation of the immune system of the skin is also present however, if the amount of the immune cells in the diseased skin is altered negligibly compared with the healthy skin, but the diseased cells can be eliminated from the body by a specific stimulation, mediated by a therapeutic agent, of the immune system. Examples thereof are tumors or other proliferative disorders of the skin.

Skin disorders which can be treated by suppression of the immune system of the skin are, for example, alopecia areata, alopecia totalis, alopecia subtotalis, alopecia universalis, alopecia diffusa, atopic dermatitis, lupus erythematosus of the skin, lichen planus, dermatomyositis of the skin, atopic eczema, neurodermatitis, morphea, scleroderma, psoriasis vulgaris, psoriasis capitis, psoriasis guttata, psoriasis inversa, alopecia areata ophiasis type, androgenetic alopecia, allergic contact eczema, irritative contact eczema, contact eczema, pemphigus vulgaris, pemphigus foliaceus, pemphigus vegetans, cicatricial mucosal pemphigoid, bullous pemphigoid, mucosal pemphigoid, dermatitis, dermatitis herpetiformis duhring, urticaria, necrobiosis lipoidica, erythema nodosum, lichen vidal, prurigo simplex, prurigo nodularis, prurigo acuta, linear IgA dermatosis, polymorphic light eruption, erythema solaris, lichen sclerosus et atrophicans, exanthema of the skin, drug rash, purpura chronica progressiva, dihidrotic eczema, eczema, fixed drug eruption, photoallergic skin reaction, lichen simplex, perioral dermatitis or graft versus host disease.

A particularly preferred use of the invasomes of the invention is the prevention and/or therapy of alopecia areata (all clinical forms), of psoriasis vulgaris (all clinical forms), of atopic dermatitis, of atopic eczema, of neurodermatitis, of polymorphic light eruption, of erythema solaris, of allergic and irritative contact eczema, of drug rash and/or of graft versus host disease.

A further use of the invasomes of the invention is the therapy of a disorder which can be treated by modulation of the immune system. A disorder which can be treated by modulation of the immune system is present if the diseased cells can be eliminated from the body by a specific stimulation, mediated by a therapeutic agent, of the immune system. Disorders which can be treated by such a modulation of the immune system comprise, for example, tumors, hyperplasias and/or other proliferative disorders, arthritis, viral diseases and bacterial and/or parasitic infections.

For this use the invasomes can also be administered in another suitable way, for example intraperitoneally or intraarticularly.

A further use of the invasomes of the invention is prophylactic and therapeutic vaccination and immunotherapy through topical application. Propylactic and therapeutic vaccinations which can be carried out with the invasomes of the invention comprise, for example, oncoses, viral diseases and bacterial and/or parasitic infections.

In addition, example 8 shows that even invasomes of the invention which are not loaded with a pharmacological agent lead to an activation of cells of the immune system, for example Langerhans cells. The invasomes of the invention are therefore particularly suitable for the transport of immunomodulating pharmacological agents because they may enhance, virtually as adjuvant, the effect of immunomodulating pharmacological agents. The present invention therefore also relates to the use of an invasome consisting of a lipid mixture, where the proportion of one or more lipids and one or more lipophosphatides in the lipid mixture is in the range from about 0.1 to about 40% by weight of the lipid mixture, as adjuvant in the treatment of disorders which can be treated by modulation of the immune system, in particular skin disorders which can be treated by modulation of the immune system.

In a preferred embodiment, the lipids are neutral, anionic or a mixture of neutral and anionic lipids.

In a preferred embodiment of the use according to the invention, the proportion by weight of the neutral, anionic or of a mixture of neutral or anionic lipids, preferably of a neutral lipid, in the lipid mixture is in the range from about 40 to about 99.9% by weight, preferably from about 50 to about 98% by weight, more preferably in the range from about 60 to about 95% by weight, even more preferably in the range from about 75 to about 95% by weight and most preferably about 90% by weight.

In a preferred embodiment of the use according to the invention, the invasomes comprise the lysophosphatides lysophosphatidylcholine, lysophosphatidylethanolamine, lysophosphatidylinositol, monolysocardiolipin, dilysocardiolipin or lysophosphatidylserine, in particular lysophosphatidylcholine. In this connection, in a preferred embodiment of the use according to the invention, the proportion of lysophosphatides in the lipid mixture is in the range from about 1 to about 25% by weight, preferably from about 4 to about 15% by weight and most preferably from about 10 to about 15% by weight.

If the invasomes are used as adjuvant, either they may include a pharmacological agent and/or the pharmacological agent can be added to the previously formed invasomes. It is thus possible to include a first pharmacological agent in the invasome and to add a second to the invasome suspension.

The following examples and the table serve merely to illustrate the inventive concept but do not restrict the subject matter of the invention.

It is evident to the skilled worker that a large number of modifications and variations of the compositions and of the methods of this invention can be carried out. It is therefore intended that this invention encompasses such modifications and variations on condition that they are within the quotation conferred by the claims and equivalents thereof.

The priority application DE 10105659.1, filed on Feb. 8, 2001, and all documents cited herein are included in the description by reference.

The following examples are intended only to describe the invention in detail without restricting it.

### EXAMPLES

1. Preparation of Invasomes

The invasomes were prepared using the alcohol solution method. A suitable amount of Phospholipon 80 (Nattermann GmbH, Germany) and ethanol (in the ratio 3:1) were mixed and vortexed for 5 minutes. Then cyclosporin A (0.5% by weight in the final mixture) and D-limonene, cineol and citral (in the ratio 10:45:45 v/v; 2% by weight of the final mixture) were added and the mixture was again vortexed. This was followed by sonication with ultrasound (Bransonic ultrasonic cleaner, Connecticut, USA) for 5 minutes. A suitable amount of phosphate buffer pH 7.4 (86.17% by weight of the final mixture in the case where no terpenes were added or 84.17% by weight of the final mixture on addition of terpenes) was added dropwise to the ethanolic mixture of lipid and cyclosporin with continued stirring until a coarse-grain suspension was obtained. These multilamellar vesicles were then sonicated or filtered, using an Avestin press (Avestin EmulsiFlex-C5, Canada), firstly for 5 min through a 200 nm polycarbonate filter, then for 10 min through a 100 mn polycarbonate filter and finally through a 50 nm polycarbonate filter until the average size of the invasomes was 50-70 nm. All the filters were obtained from Costar (Costar Nucleopore Polycarbonate, USA). The diameter of the invasomes was determined using a Zetasizer IV (Malvern Instruments, Malvern UK). The diameter of the invasomes was in the range 50-70 nm. Subsequently, the polydispersity index (PI) was assessed as a measure of the homogeneity of the resulting invasome preparation. The PI for the invasomes was below 0.3. This result indicates a homogeneous invasome population.

2. Treatment of DEBR Rats With Invasomes

15 DEBR rats with an average age of 19 months (the age was within the range 14-26 months) and with a weight in the range 200-350 g were used in the experiment. The rats were kept singly in cages with water and food ad libitum. The observed hair loss extended from large bare patches on the flanks of the animals and hairless areas on the head up to almost complete loss of all hair on the body and head. The rats were divided into 3 groups each of 5 animals with similar hair loss (group I, II and III). The experiments were carried out with the two invasome types described above with or without added terpenes. The invasome control was prepared in the same way but without cyclosporin A. The liposomal preparations were applied nonocclusively to the backs (dorsal side) of the rats. A defined area of 2×2 cm was marked on the backs of the rats for the application.

Group I was used to investigate the activity of the cyclosporin-loaded invasomes containing D-limonene, citral and cineol on hair growth. 20-80 μl/cm² of the invasome preparation was applied to a marked area 2×2 cm in size on one of the bare flanks of each rat twice a day for 7 weeks, while the opposite (contralateral) flank was treated with the control preparation.

Group II was used to investigate the activity of cyclosporin-loaded invasomes on hair growth. 20-80 μl/cm² of the invasome preparation was applied to a marked area 2×2 cm in size on one of the bare flanks of each rat twice a day for 7 weeks, while the opposite (contralateral) flank was treated with the control preparation. 20-80 μl/cm² of an ethanolic cyclosporin A solution (0.5%) were applied within the marked area 2×2 cm in size on one of the bare flanks of rats in group III twice a day for 7 weeks, while the contralateral flank remained untreated.

The morphological changes were examined each day. Before the treatment, all the rats in group I and II had large bare patches in the abdominal, dorsal, head and shoulders areas. All 5 rats in group I showed hair growth on the treated site 7 days after the start of treatment, whereas no hair growth was to be observed on the treated control area. On the 21st day after the start of treatment, 4 of the 5 rats showed sparse to moderate hair growth, and after 36 days the fur had regained the normal density on the treated site. Whereas hair growth continued on the treated site, further hair loss was to be observed at other sites on the skin. This indicates that the effect of cyclosporin A was confined only locally to the application site, and no or only a slight systemic effect of cyclosporin occurred. This was also indicated by the fact that in an HPLC analysis of the rat serum no cyclosporin A was detectable in the blood 14 hours after the first treatment or at the end of the study either. One of the 5 rats showed normal hair growth on the whole body after only 21 days but an enhanced effect on the treated site. This observation may be explained, for example, by the fact that 2-3% of DEBR rats show spontaneous regrowth of all hair even without treatment.

In the 5 rats in group II, first signs of hair growth were observed for all the rats 14 days after the start of the treatment, whereas no hair growth was observable on the control areas. Hair growth continued within and immediately adjacent to the application areas, and weak to moderate hair growth was observed on the treated regions on the 21st day. Hair growth continued and led to a normal density of fur at the treatment site after about 42 days. 14 hours after the start of the treatment and at the end of the treatment it was not possible to detect cyclosporin A in the rats' blood by HPLC analysis. Once again, one of the rats showed hair growth over the whole body as already observed for the rats in group I.

The rats in group III showed no hair growth throughout the treatment period; on the contrary, the hair loss continued at other sites on the body for all the rats in this group.

The results described above are summarized in table 1.

3. HPLC Analysis

The HPLC system used was a Merck-Hitachi HPLC 655 A-12 liquid chromatography pump equipped with a Kontron 360 autosampler and with a Merck-Hitachi L-5000 LC control unit. The column used was a LiChrospher 100 RP-18 column which contained spherically shaped 5 μm silica gel particles (3 mm I.D. and 125 mm length). The UV detector was a Merck-Hitachi L-4000 UV absorption detector. The integrator used was a Merck-Hitachi D-7500 integrator. The liquid phase used for the elution was methanol:water:acetonitrile in the ratio 10:30:60. The flow rate was 0.7 ml/min, with the column being kept at a temperature of 75° C. The pressure used was 43-44 bar. UV detection took place at 208 nm. The retention time for cyclosporin A was 8.65 minutes.

A standard cyclosporin solution was used to calibrate the system, the detection limit being about 50 ng/ml. A linear relation between absorption and concentration was observed over the entire calibration range up to 7000 ng/ml cyclosporin A.

4. Effect of Terpenes on the Membrane of Invasomes

The invasomes were prepared as described under 1. but only 0.35% by weight cyclosporin A was used in place of 0.5%. Addition of the terpene mixture consisting of 45% cineol, 45% citral and 10% D-limonene comprised 0, 0.25, 0.5, 0.75, 1.0, 1.25, 1.5 or 2% by weight. Phosphate-buffered saline solution (PBS) was added appropriately for the added amount of terpene and cyclosporin A up to 100% by weight. The suspension was then extruded in an Emulsiflex C5 successively through 400 nm, 200 nm, 100 nm and 50 nm polycarbonate membranes with the homogenizer slit open for 30 minutes in each case. The diameter of the invasomes was determined with a Zetasizer IV.

The size of the invasomes prepared in this way, containing a 2% terpene mixture, was:

| | |
|---|---|
| Invasomes | 87.6 ± 0.5 nm |
| Invasomes with terpenes | 85.1 ± 0.4 nm |
| Cyclosporin invasomes | 74.1 ± 0.8 nm |
| Cyclosporin invasomes with terpenes | 108.4 ± 0.9 nm |

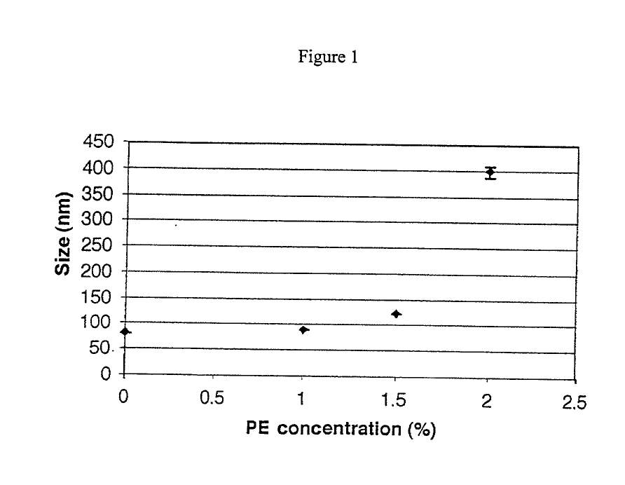

The invasomes were prepared as described above. All the amounts/volumes employed were reduced to 1/10. The emulsions were extruded with a LiposoFast from Avestin successively through a 400 nm, 200 nm, 100 nm and 50 nm filter 21 times in each case. The concentration of the terpenes was varied as described above. The size of the invasomes as a function of the proportion by weight of the terpene mixture is depicted in FIG. 1.

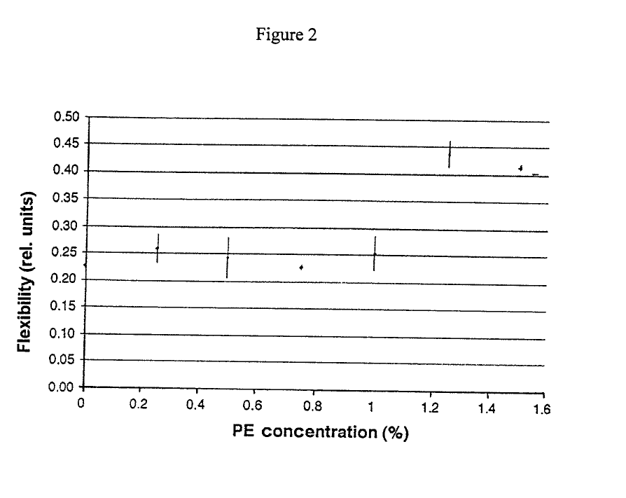

In a further experiment, the invasome membrane was labeled with the fluorophore DPH (diphenylhexatriene) in a molar ratio of 1:400 (DPH/lipid), and the anisotropy of the membrane was determined as a function of the terpene concentration by fluorescence spectroscopy. The result is depicted in FIG. 2.

Cyclosporin A invasomes without terpene mixture had a size of 79.9±0.56 mm after manual extrusion. As the terpene concentration increased, the size of the invasomes after manual extrusion grew (FIG. 1). It was 398.6±10.8 nm with 2% terpene and extrusion through a 50 nm filter. This shows that PV leads to a flexibilization of the membrane.

The anisotropy, a measure of the fluidity of the membrane, increased at concentrations above 1% by weight terpene mixture from 0.25 rel. units to about 0.4 rel. units (FIG. 2). This means that the terpenes make the membrane more flexible.

5. Penetration of Cyclosporin A Invasomes into the Human Skin Ex Vivo

The invasomes were prepared as described under 4., using 25 μl of fluorescence-labeled cyclosporin A (2 mmol in DMSO) and unlabeled cyclosporin A. 1% by weight terpene mixture was added to an invasome preparation. The solution was hydrated by agitating (Vortex) with 0.5 ml of PBS (corresponding to about 83% by weight). The suspensions were then extruded through a 400-50 nm filter cascade using a hand extruder (LiposoFast, Avestin). The final lipid concentration was 10% and the cyclosporin concentration was about 0.35%. The invasomes or an ethanolic solution of fluorescence-labeled cyclosporin as control were applied nonocclusively to human abdominal skin for 6 h. The skin samples were embedded in Tissue Tec, and 7 μm-thick transverse sections through the skin were prepared with a Cryo-cut (−25° C.). These were analyzed in a confocal microscope with maximum laser power, open pin hole and with gain and offset kept constant.

Fluorescence-labeled cyclosporin applied in ethanolic solution is located only in the topmost layers of the epidermis (stratum corneum). Fluorescence-labeled cyclosporin in invasomes without terpene mixture is detectable in the epidermis and in lower concentrations in the dermis. Aggregates of fluorescence-labeled cyclosporin are visible on/in the stratum corneum in this case too. Invasomes with 1% terpene mixture and fluorescence-labeled cyclosporin A are homogeneously distributed in the stratum corneum, and fluorescent cyclosporin A is detectable in the dermis. This means that the terpene mixture in a concentration of 1% leads to a homogeneous distribution of cyclosporin A in the epidermis and enhances penetration into the deeper layers of skin.

6. Effect of Lysophosphatidylcholine on the Penetration of Invasomes into Human Skin Ex Vivo

1.75 mg of cyclosporin A, 66.6 mg of ethanolic lipid solution and 10 μCi of ³H-cyclosporin (10 μl of ethanolic solution with a spec. activity of 296 GBq/mmol) were weighed into a round-bottomed flask. The following lipids and lipid mixtures were used in this case:

100% soybean PC (99% purity)

94% soybean PC+6% lyso-PC

90% soybean PC+10% lyso-PC

85% soybean PC+15% lyso-PC

100% Phospholipon 80 (soybean lipid with about 75% PC, about 5% lyso PC and about 20% other lipids)

100% Phospholipon 100 (purified soybean lipid which contains only phosphatidylcholine and lysolecithin)

Soybean phosphatidylcholine (soybean PC) and lysophosphatidylcholine (lyso-PC) were purchased from Sigma-Aldrich (Seelze) and Phospholipon 80 (PL 80) and Phospholipon 100 (PL 100) were purchased from Nattermann. If the lipids were dissolved in chloroform, the chloroform was removed in a rotary evaporator and the lipid film was dried. An ethanolic lipid solution was prepared by adding ethanol. The mixture was vortexed for 5 min and sonicated in an ultrasonic bath for 20 min until the cyclosporin was completely dissolved. The solution was then hydrated by slow addition of 0.431 ml of PBS while vortexing and was incubated in the dark at room temperature for 30 min. The resulting suspension was extruded through polycarbonate filters with a pore diameter of 400, 200, 100 and 50 nm 21 times each. 30 μl per cm² of this emulsion were applied nonocclusively to human abdominal skin clamped in a Franz diffusion cell and incubated for 6 h. All the mixtures were repeated 3-6 times. Subsequently the surface of the skin was wiped off with ethanol, and the horny layer of the epidermis was removed with 20 Tesa film strips. The thickness of the stratum corneum strip was calculated from the weight of the Tesa film strip. The epidermis was then cut into 30 μm-thick layers and the individual samples were extracted in methanol/acetic acid overnight. After addition of RiaLuma, the radioactivity of the samples was determined for 40 min or with an error tolerance of ≦2%.

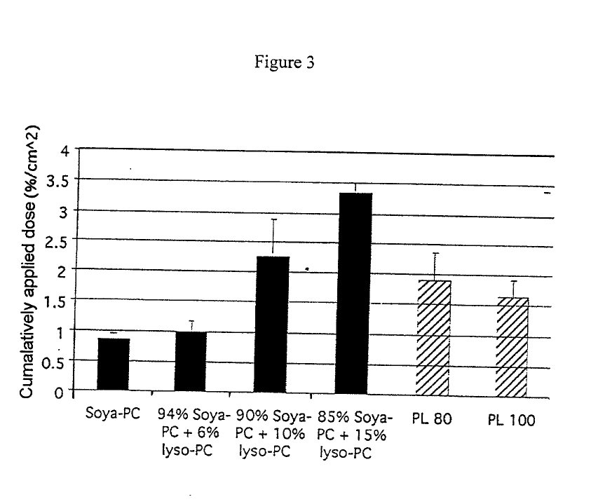

As the lyso-PC concentration in liposomes composed of soybean lipid increases there is an increase in the penetration of cyclosporin into the stratum corneum 1 5 (FIG. 3). Whereas only 0.85±0.11% of the applied cyclosporin is in the stratum corneum 6 h after application of pure soybean lipid liposomes, the amount increases to 3.35±0.42% when 15% lyso-PC is present in the liposomes. Cyclosporin invasomes composed of the natural soybean lipid extract Phospholipon 80 transport 1.92±0.44% of the applied dose into the stratum corneum and invasomes composed of the natural soybean lipid Phospholipon 100, which consists only of phosphatidiylcholine and lysolecithin, transport 1.65±0.27% into the stratum corneum. The ratio of lysophosphatidylcholine to phosphatidylcholine in these natural extracts is 1:10. Liposomes composed of 90% phosphatidylcholine and 10% lysophosphatidylcholine penetrate into the stratum corneum just as well or better (2.27±0.10%) as liposomes composed of the natural extracts Phospholipon 80 and Phospholipon 100.

It follows from this that lysophosphatidylcholine improves penetration of the invasomes of the invention into the skin.

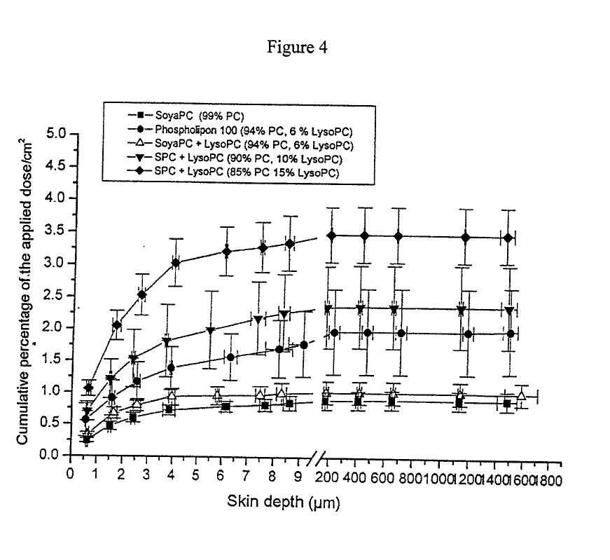

The same picture is obtained when the cumulative amount of the topically applied cyclosporin A in the individual layers of skin is determined (FIG. 4). Once again, it is evident that liposomes with 15% lysophosphatidylcholine penetrate into the epidermis best. As the lyso-PC concentration falls, the penetration decreases. Invasomes composed of the natural extracts Phospholipon 80 or Phospholipon 100 increase the penetration of cyclosporin A into the epidmeris to the same extent as liposomes composed of soybean PC with the addition of 10% lyso-PC.

Table 2 summarizes the amounts of cyclosporin A accumulated in the individual layers of skin. Cyclosporin A is detectable only in traces in the receiver compartment underneath the skin. This shows that cyclosporin A does not penetrate to a significant extent through the skin. All of the topically applied cyclosporin A was recovered (see last column).

7. Penetration of Cyclosporin A Invasomes Through the Skin in an Animal Model

Quantification of ³H-labeled cyclosporin A in the blood and organs after topical application of invasporin

There DDAB rats were treated on an area of 4 cm² with 80 μl of a suspension of invasomes composed of NAT-8539 (ethanolic soybean lipid solution with a composition like Phospholipon 80) containing 3.5 mg/ml cyclosporin and 0.5% terpene mixture. The applied amount of cyclosporin A was 280 μg with a total activity of 9.6 μCi (specific activity: 34.3 μCi per mg of cyclosporin A). 100 μl retroorbital blood samples were taken at various times (t=0, 2 h, 4 h, 6 h, 9 h, 24 h, 35 h, 48 h) for up to two days after application. After 48 h, the liver, spleen and both kidneys were removed, and tissue samples (about 200 mg) were prepared for the cyclosporin A determination (liver quadruplicate. spleen and kidneys each duplicate determination). After processing of the blood and tissue samples (incubation with perchloric acid and hydrogen peroxide at 60° C. overnight), the ³H activity of the samples was measured.

The blood samples from all three rats did not contain a significant amount of radioactivity at any time. All the values were distinctly below the limit of detection, which was determined by a calibration curve as under 3. and was 4 ng/ml of cyclosporin A (0.4 ng per 100 μl of blood sample is equivalent to 30 dpm after subtraction of the 30 dpm background activity).

The maximum levels in the organs (see table 3) were 38 ng/g of liver tissue (rat 1: 323 ng/complete organ), 12 ng/g of spleen tissue (rat 1: 13 ng/complete organ) and 14 ng/g of kidney tissue (rat 1: 25 ng in both kidneys). This corresponds to a maximum percentage content of 0.13% of the applied dose in these three organ systems.

8. Activation of Langerhans cells by Empty Invasomes

Preparation of epidermal sheets and immunohistochemistry:

Empty invasomes prepared as described under 1 but with cyclosporin A were applied to the ears (50 μl/ear) of anesthetized mice. The animals were sacrificed after 16 h and the ears were detached. The ears were shaved to remove the hair and then pulled apart along the ear cartilage with forceps to result in two halves of the ear (each consisting of dermis and epidermis). The halves of the ear were then incubated in a 20 mM EDTA solution at 37° C. for 1 h and subsequently the epidermis was stripped off from the dermis. The epidermis then looked like a thin membrane (epidermal sheet), which was fixed in acetone (−20° C.) for 10 min. The Langerhans cells (LC) were then visualized immunohistochemically by means of the peroxidase technique. LCs were detected using an anti-MHC-II antibody because strong MHC-II expression is characteristic of all LCs and this is modulated to only a minor extent by activation. An anti-CD86 antibody was used to demonstrate LC activation because this surface antigen is upregulated markedly (about 100-fold) during LC activation. Finally, the epidermal sheets were drawn onto slides, and a nuclear staining and microscopic evaluation were carried out.

Preparation of a single cell suspension (SCS) from mouse ears:

Halves of ears were prepared as described above. Two halves of ears were then incubated in 1 ml of an enzyme solution (collagenase III, dispase, DNase; 0.4 mg/ml; 0.2 mg/ml; 0.16 mg/ml) at 37° C., shaking occasionally, for 30 min, and then the epidermis was stripped off the dermis, and a single cell suspension was prepared by vigorous pipetting up and down. The number of cells from one ear was in this case between 3 and 7×10⁵ cells. This is a sufficient number of cells for up to 4 different (dual) stains for FACS analysis.

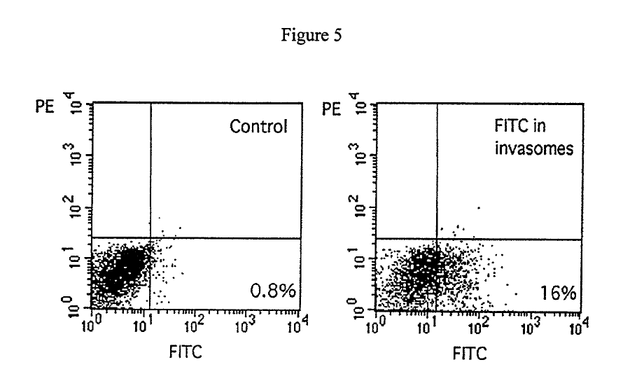

Invasomes (formulated from Phospholipon 80) with encapsulated fluorescein isothiocyanate (FITC; Sigma-Aldrich; Seelze) were prepared as described under 1. However, 0.35% by weight FITC was used in place of 0.5% by weight cyclosporin A. FITC-invasomes and an ethanolic FITC solution for comparison were applied topically to mouse ears. 16 hours later, both epidermal sheets and single cell suspensions were prepared as described above, and the uptake of FITC into epidermal cells was analyzed. After application of FITC-invasomes, 16% of the cells were FITC-labeled (see the FACS analysis in FIG. 5), whereas FITC not encapsulated in invasomes is unable to penetrate into the skin to a significant extent. This unambiguously suggests that there is efficient penetration of FITC-invasomes into the skin and uptake thereof and of their constituents by epidermal cells.

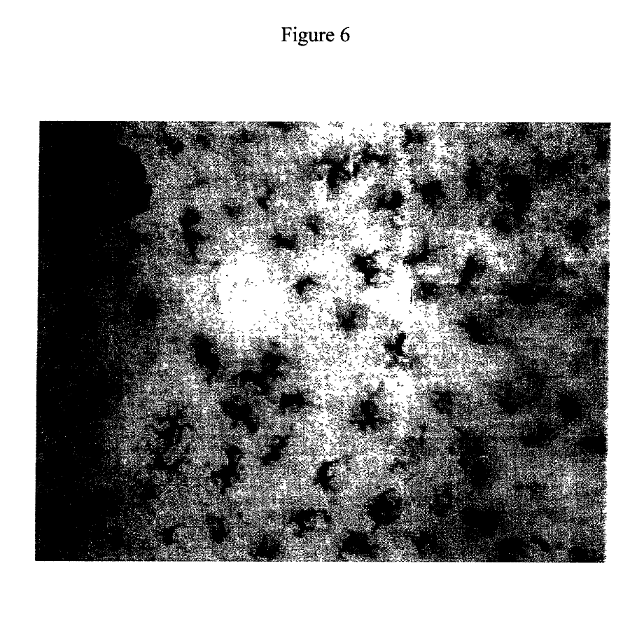

Empty invasomes (formulated from Phospholipon 80) activated LCs after topical application to a considerable extent, which was demonstrable by the upregulation of CD86 in epidermal sheets (table 4 and FIG. 6).

TABLE 1

| | | age of the rat | | | | | | |
|---|---|---|---|---|---|---|---|---|
| Rat | | at the start | Hair growth status | | | | | |
| No. | sex | of the experiment | day 0 | day 7 | day 14 | day 21 | day 35 | day 42 |
| 2-I | F | 14 | − | −/+ | +/++ | ++ | ++/+++ | ++/+++ |
| 7-I | F | 21 | − | + | ++ | ++/+++ | +++ | +++ |
| 8-I | F | 21 | − | −/+ | + | +/++ | +/++ | ++ |
| 9-I | F | 21 | − | −/+ | + | +/++ | +/++ | ++ |
| 10-I | F | 21 | − | −/+ | +/++ | ++ | ++/+++ | ++/+++ |
| 1-F | F | 14 | − | − | + | ++ | ++/+++ | +++ |
| 4-F | F | 21 | − | −/+ | + | +/++ | +/++ | +/++ |
| 6-F | F | 21 | − | − | −/+ | ++ | ++ | ++/+++ |
| 13-F | M | 18 | − | − | −/+ | + | ++ | +++ |
| 14-F | M | 18 | − | − | −/+ | + | +/++ | ++ |
| 3-C | F | 14 | ++ | ++ | ++ | ++ | ++ | + |
| 5-C | F | 26 | +/++ | +/++ | +/++ | +/++ | +/++ | + |
| 11-C | M | 24 | ++ | ++ | ++ | ++ | ++ | + |
| 12-C | M | 18 | ++ | ++ | ++ | ++ | ++ | ++ |
| 15-C | F | 18 | +/++ | +/++ | +/++ | +/++ | +/++ | +/++ |

TABLE 2

| | Cyclosporin | Cyclosporin | Cyclosporin | Cyclosporin | Total |
|---|---|---|---|---|---|
| Liposomes or | on the | in the stratum | in deeper | in receiver | recovered |
| invasomes | surface | corneum | skin | compartment | cyclosporin A |
| Soya PC | 100.32 ± 1.60 | 0.85 ± 0.11 | 0.016 ± 0.001 | 0.0014 ± 0.0009 | 101.23 ± 1.59 |
| Phospholipon 100 | 96.71 ± 3.05 | 1.65 ± 0.27 | 0.179 ± 0.074 | 0.0019 ± 0.0007 | 98.55 ± 3.05 |
| Soya PC + | 104.77 ± 2.72 | 0.99 ± 0.16 | 0.023 ± 0.012 | 0.00046 ± 0.0002 | 105.79 ± 2.77 |
| lyso-PC (94:6) % | | | | | |
| Soya PC + | 94.88 ± 2.27 | 2.27 ± 0.59 | 0.10 ± 0.064 | 0.0023 ± 0.0008 | 97.24 ± 1.26 |
| lyso-PC (90:10) % | | | | | |
| Soya PC + | 94.56 ± 2.94 | 3.35 0.42 | 0.14 ± 0.009 | 0 | 98.05 ± 2.83 |
| lyso-PC (85:15) % | | | | | |
| Phospholipon 80 | 104.76 ± 1.71 | 1.92 ± 0.44 | 0.041 ± 0.014 | 0.0056 ± 0.00019 | 106.73 ± 1.95 |

TABLE 3

| | | ng Cyclosporin A | | |
|---|---|---|---|---|
| | | per g | per organ | % of dose |
| Rat 1 | liver | 38.3 | 323.1 | 0.1154 |
| | spleen | 12.4 | 13.1 | 0.0047 |
| | left kidney | 13.0 | 11.2 | *0.0091 |
| | right kidney | 15.7 | 14.2 | |
| | | | | Σ 0.1292 |
| Rat 2 | liver | 24.6 | 199.4 | 0.0712 |
| | spleen | 6.4 | 5.3 | 0.0019 |
| | left kidney | 5.8 | 5.4 | *0.0032 |
| | right kidney | 4.4 | 3.6 | |
| | | | | Σ 0.0763 |
| Rat 3 | liver | 32.4 | 272.9 | 0.0975 |
| | spleen | 7.2 | 5.8 | 0.0021 |
| | left kidney | 8.2 | 7.8 | *0.0051 |
| | right kidney | 7.3 | 6.4 | |
| | | | | Σ 0.1047 |

*Cumulative value for both kidneys

TABLE 4

| | CD86-positive cells per microscopic |
|---|---|
| Formulation | field |
| None (control) | 2 |
| Invasomes | 20 |
| FITC-invasomes | 19 |

1. An invasome comprising:
a) a lipid mixture comprising one or more lipids and one or more lysophosphatides, where the proportion of lysophosphatides in the lipid mixture is in the range from 0.1 to 40% by weight; and
b) at least one pharmacological agent.

2. The invasome as claimed in claim 1, where the lipids are selected from the group consisting of neutral lipids, anionic lipids and a mixture of neutral and anionic lipids.

3. The invasome as claimed in claim 2, where the proportion of neutral or anionic lipids or a mixture of neutral and anionic lipids in the lipid mixture is in the range from 40 to 99.9% by weight.

4. The invasome as claimed in claim 1, where the invasome comprises the lipid mixture and the pharmacological agent in a ratio of from 1:1 to 1 000:1, preferably from 2:1 to 100:1, by weight.

5. The invasome as claimed in claim 1, where the invasome has a diameter of from 30 to 400 nm.

6. The invasome as claimed in claim 1, where the lipid mixture is obtained from sources selected from the group of soybeans, cotton seeds, coconut kernel, peanut, safflower seeds, sesame seeds, sunflower seeds, linseeds, oilseed rape, wheatgerms, olives, whale fat, stratum corneum lipid, neatsfoot oil and egg.

7. The invasome as claimed in claim 1, where the proportion of neutral lipids in the lipid mixture is in the range from 75 to 95% by weight.

8. The invasome as claimed in claim 1, where the neutral lipids are selected from the group consisting of glycerophospholipids, in particular phosphatidyl-cholines, steroids, glycerophosphonolipids, glycerophosphinolipids and sphingolipids.

9. The invasome as claimed in claim 1, where the lysophosphatides are selected from the group consisting of lysophosphatidylcholines, lysophosphatidyl-ethanolamines, lysophosphatidylinositol, monolysocardiolipin, dilysocardiolipin and lysophosphatidylserines.

10. The invasome as claimed in claim 1, where the invasome comprises one or more terpenes.

11. The invasome as claimed in claim 10, where the terpene is selected from the group consisting of cineol, citral, limonene, in particular D-limonene, menthane, terpinene, terpinolene, menthol, in particular 1-menthol, carveol, in particular 1-carveol, menthone, carvone, pinene, in particular β-pinene, carene, in particular 3-carene, terpineol, terpinen4-ol, pulegone, piperitone, cyclohexane oxide, limonene oxide, pinene oxide, cyclopentene oxide, ascaridol, 7-oxybicyclo[2.2.1]heptane, cymene, camphene, citronellol, geraniol, nerol, linalool, borneol, thujol, sabinol, myrtenol, thymol, verbenol, fenchol, piperitol, perillaaldehyde, phellandral, citronellal, myrtenal, piperitone, thujone, umbellulone, verbenone, chrysanthenone, fenchone, camphor, quinone, menthofuran, linalool oxide, rose oxide and qinghaosu.

12. The invasome as claimed in claim 1, where the pharmacological agent is selected from the group consisting of an immunosuppressant, an immunostimulant, an antiallergic, an antibiotic, an antiinfective, a cytostatic, a cytotoxic agent, a mitogen, a chemokine, a cytokine, a dernatic and a physiological or pharmacological inhibitor of a mitogen, of a chemokine or of a cytokine.

13. The invasome as claimed in claim 12, where the immunosuppressant is selected from the group consisting of a glucocorticoid, in particular beclomethasone, betamethasone, clocortolone, cloprednol, cortisone, dexamethasone, fludrocortisone, fludroxycortide, flumetasone, fluocinolone acetonide, fluocinonide, fluocortolone, fluorometholone, fluprednidene acetate, hydrocortisone, paramethasone prednisolone, prednisone, prednylidene, pregnenolone, triamcinolone or triamcinolone acetonide, a cyclosporin, in particular cyclosporin A, mycophenolate mofetil, tacrolimus, rapamycin, FK 506, cycloheximide-N-ethyl ethanoate, azathioprine, ganciclovir, an anti-lymphocyte globulin, ascomycin, myriocin, pharmacological inhibitors of MAP kinases and methotrexate.

14. The invasome as claimed in claim 1, where the pharmacological agent is selected from the group consisting of a nucleic acid, a protein, a peptide, a sugar and a lipid.

15. The invasome as claimed in claim 14, where the nucleic acid is selected from the group consisting of an antisense oligonucleotide, an antisense RNA, an RNAi, an siRNA and an oligonucleotide which forms a triple helix.

16. A method for preparing an invasome as claimed in claim 1, characterized in that the lipid mixture is mixed with at least one pharmacological agent.

17. A medicament comprising an invasome as claimed in claim 1 and suitable excipients and additives.

18. A method for using an invasome as claimed in claim 1 for the therapy of a skin disorder.

19. The method as claimed in claim 18, where the skin disorder can be treated by modulation of the immune system.

20. The method as claimed in claim 19, where the skin disorder is selected from the group consisting of alopecia areata (all clinical forms), psoriasis vulgaris (all clinical forms), atopic dermatitis, atopic eczema, neurodermatitis, polymorphic light eruption, erythema solaris, allergic and irritative contact eczema, drug rash and graft versus host disease.

21. A method for using an invasome as claimed in claim 1 for the therapy of a disorder which can be treated by modulation of the immune system, in particular for a prophylactic and/or therapeutic vaccination.

22. The method as claimed in claim 21, where the disorder is selected from the group consisting of an oncosis, a hyperplasia, a proliferative disorder, an arthritis, a viral disease, a bacterial and/or parasitic infection.

23. A method for using an invasome comprising a lipid mixture comprising one or more lipids and one or more lysophosphatides, where the proportion of lysophosphatides in the lipid mixture is in the range from 0.1 to 40% by weight, as adjuvant and/or carrier system for antigens and/or other immunomodulating molecules in the treatment of a disorder which can be treated by modulation of the immune system.
